(19) United States
(12) Patent Application Publication
(10) Pub. No.: US 2013/0324865 A1
(43) Pub. Date: Dec. 5, 2013

(54) DEVICE FOR MEASURING ARTERIAL PRESSURE

(71) Applicants: Igor Yavelov, Moscow (RU); Vladimir Hudoley, Pos. Timiryazevo (RU); Sergey Vlasov, Moscow (RU)

(72) Inventors: Igor Yavelov, Moscow (RU); Vladimir Hudoley, Pos. Timiryazevo (RU); Sergey Vlasov, Moscow (RU)

(21) Appl. No.: 13/821,234

(22) PCT Filed: Sep. 7, 2011
(86) PCT No.: PCT/RU11/00682

§ 371 Date: May 23, 2013

(30) Foreign Application Priority Data
Sep. 9, 2010 (RU) 2010137514

(51) Int. Cl. A61B 5/00; A61B 5/021; A61B 5/022

(52) U.S. Cl. CPC A61B 5/681; A61B 5/022; A61B 5/7225; A61B 5/741; A61B 5/02108; A61B 5/0004; A61B 5/0022
USPC 600/494

(57) ABSTRACT

A device is sued for measuring arterial pressure. The device includes a body in the form of a wrist-watch, an element for fastening the latter on a user's wrist, said element being in the form of a wristband designed so that the clamping force of the wristband on the wrist can he adjusted. A pulsed wave sensor is arranged on the element for fastening the body on the user's wrist. The device includes an analog-digital converter, a filter for isolating the alternating component of a signal, and a measurement data indicator, a voice synthesizer and a pulsed wave sensor position indicator, which are integrated by their inputs.

### FIELD OF THE INVENTION

The present invention relates to the area of medicine and medical equipment and can be used for noninvasive measuring of arterial blood pressure by sensing pressure waveform data.

### BACKGROUND OF THE INVENTION

Prior to the development of this invention, blood pressure was measured by sensing pressure waveform data using a blood pressure measurement device comprising a base unit secured on the wrist by a strap, a sensing unit sensing blood pressure, the means for converting pressure into electric signal, and the means for data collection (Russian Federation Patent No. 2140187, Class A61B 5/022 of Nov. 7, 1994).

A disadvantage of this device is its usability. Specifically, the left hand must be on the table or in suspension, whereas the right hand carries out the work with a monitor. The device is complex and contains many parts, reducing its reliability and measuring accuracy.

The design most similar in terms of essential features is a blood pressure measurement device comprising a base unit in the form of a watch containing the sensor, which is applied to the wrist of a patient. The device also includes a pressure waveform sensor, signal amplifier, filter for isolation of the signal variable component, analog-to-digital converter, voice synthesizer, and sensor location indicator (Russian Federation Patent No. 2342067, Class A61B 5/022 of Dec. 27, 2008).

The aforementioned device is small and relatively convenient to use.

A disadvantage of this device, however, is its insufficient usability. In particular, when using this device to measure blood pressure, the left hand must be positioned only either on the table or in suspension (hand at the heart level), reducing its measuring accuracy.

### SUMMARY OF THE INVENTION

The aim of the present invention is to serve as a convenient and reliable device for measuring blood pressure, ensuring the high accuracy of the measurements.

The technical results of the invention are usability and reliability in measuring blood pressure while producing highly accurate measurements.

The technical results are achieved because in addition to the following parts of the device: a base unit in the form of a watch, fastening element in the form of a bracelet which allows to fasten the unit to the wrist of the patient and the tightness of which can be regulated, analog-to-digital converter, filter for the isolation of the signal variable components, the means for data collection with integrated inputs, voice synthesizer, and sensor location indicator; the blood pressure measuring device also contains a transducer, whose processor is connected to the antenna output of the transducer, while the input of the transducer is connected to the output of the analog-to-digital converter, and a signal receiver, which looks like a mobile phone and contains the data collection indicator, voice synthesizer, and sensor location indicator, the joint inputs of which are connected via interface cables with the antenna input of the signal receiver.

It is preferable that the pressure waveform sensor contain a fiber optic transmitter.

The advantage of aforementioned invention is its usability, reliability, and accuracy for measuring blood pressure. The addition to the blood pressure measuring device of a transducer, which has a processor connected with the transducer antenna output, while the transducer input is connected to the analog-to-digital converter output, and a signal receiver, which looks like a mobile phone and contains the data collection indicator, voice synthesizer, and sensor location indicator, the joint inputs of which connected via interface cables to the the antenna input of the signal receiver, increases its reliability and usability because it allows the user to control pressure waveform sensor and to measure the blood pressure at any position of the arm, to the wrist of which the pressure waveform sensor is affixed. The fiber optic transmitter in the sensor increases the accuracy of the measurements of the blood pressure by increasing the accuracy of the sensor positioning above the radial artery.

### BRIEF DESCRIPTION OF THE DRAWINGS

FIG. 1 is a perspective view of the device for blood pressure measuring.

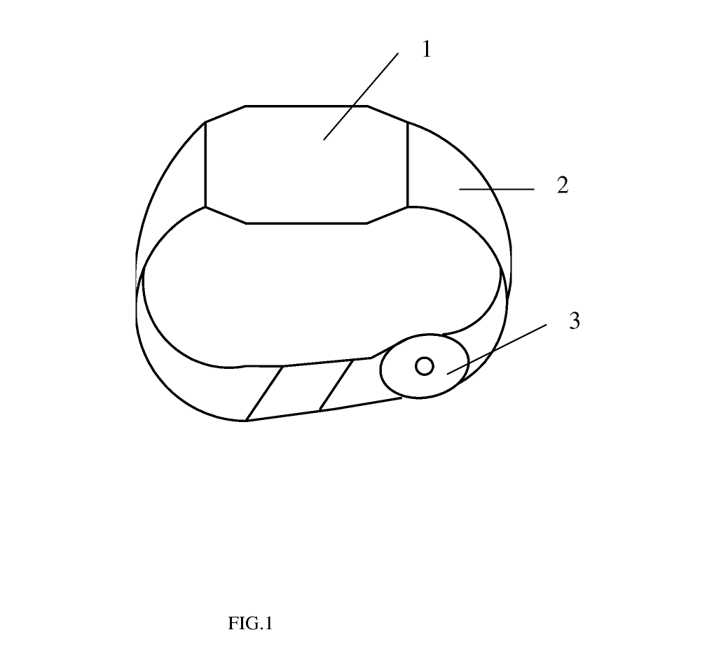

FIG. 2 is a schematic functional block diagram of a device for blood pressure measuring.

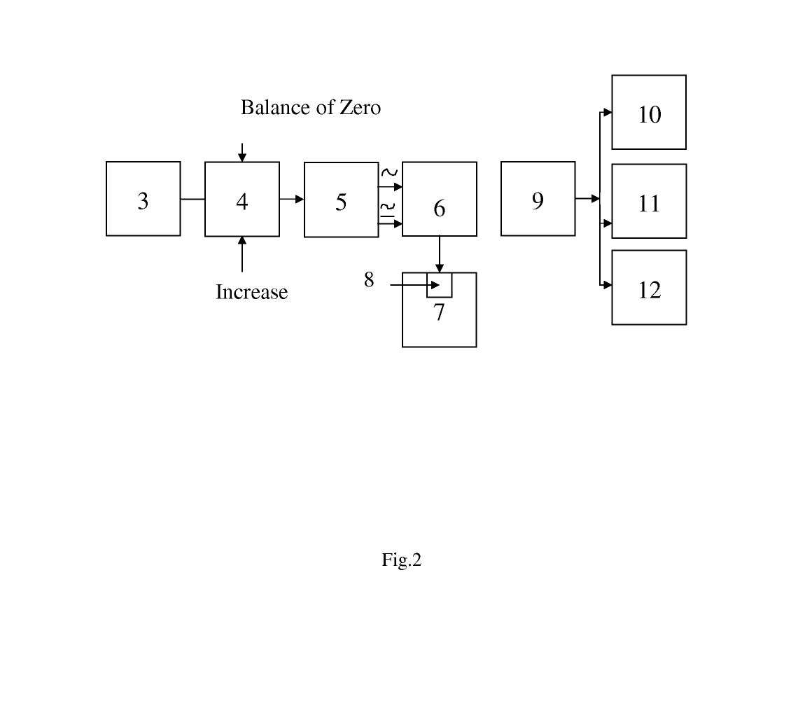

### DETAILED DESCRIPTION OF THE INVENTION

The device consists of a base unit (1) in the form of a watch affixed to the wrist with the help of a bracelet (2). The tightness with which the bracelet is secured on the wrist can be adjusted by the number of bracelet links (not depicted on the drawings).

The pressure waveform sensor (3) is located on the bracelet.

The device contains a signal amplifier (4), a filter (5) for the isolation of the signal variable component, analog-to-digital converter (6), and transducer (7) with the processor (8). A signal receiver, which looks like a mobile phone (9), contains the data collection indicator (10), voice synthesizer (11), and sensor location indicator (12).

The device operates in the following way.

Before measuring blood pressure, the user puts the bracelet on the wrist. The positioning checking occurs by turning device on when the pressure waveform (3) sensor location indicator (12) lights up. If the light is red, the axis of the sensor (3) is located above the radial artery. The shift of the cantilever element of the sensor (3) (is not depicted on the drawings) is registered by the fiber optic transmitter of the sensor (3) (is not depicted on the drawings). The electric signal determines the form of the pressure waveform, which is depicted by the data collection indicator (10). The form of the pressure waveform is the applied pressure function and is used for further processing.

The signal received by the sensor (3) is increased by the signal amplifier (4); its variable component is isolated by the filter (5) and is then converted from analog to digital form, using analog-to-digital converter (6).

The radio signal that has been received by the transducer (7) is transmitted via the transducer (7) output antenna. The transmitted signal is received via the input antenna of the signal receiver (9). The aforementioned operations are controlled by the processor (8). The signal receiver is in the form of the mobile phone, e.g., Nokia 6230, which contains the data collection indicator (10), voice synthesizer (11), and sensor location indicator (12). This set up allows receiving visual and audio information regarding the pressure waveform, while there is no wire connection between the base unit (1) and the receiver (9). This allows to measure blood pressure at any position of the left hand while maintaining the accuracy of the measurements.

The use of the device for measuring blood pressure described above allows the users to measure their blood pressure in any conditions. The user can measure blood pressure at any position of the hand with the base unit that contains the pressure waveform sensor attached to the wrist; the location of the data collection indicator, voice synthesizer, and sensor location indicator in the mobile phone that is wirelessly connected to the pressure waveform sensor tremendously increases the usability of the device and the control over its accurate position relative to the wrist. Thus, the invented device that has been described above will be widely used in medicine for measuring blood pressure, both at hospitals and at home.

1. (canceled)

2. (canceled)

3. A device for measuring arterial pressure of a human, said device comprising:
a body in the form of a wrist-watch;
an element for fastening said body on a wrist of the human, said element being in a form of a wristband designed so that clamping force of said wristband on the wrist can be adjusted;
a pulsed wave sensor arranged on said element for fastening said body on the wrist of the human;
an analog-digital converter, a filter for isolating the alternating component of a signal, and a measurement data indicator, a voice synthesizer and a pulsed wave sensor position indicator, which are integrated by their inputs; and
a signal transmitter provided with a processor which is connected to an antenna output of the signal transmitter, the input of the signal transmitter being connected to the output of the analog-digital converter, and a signal receiver, which is in the form of a cellular telephone.

4. A device for measuring arterial pressure of a human as set forth in claim I wherein said measurement data indicator, said voice synthesizer and said pulsed wave sensor position indicator are arranged in said body of the cellular telephone, and the integrated inputs thereof are also connected via an interface cable to an antenna input of the signal receiver.
